(19) United States
(12) Patent Application Publication
(10) Pub. No.: US 2026/0076322 A1
(43) Pub. Date: Mar. 19, 2026

(54) BRASSICA CYTOPLASMIC MALE STERILITY (CMS) FERTILITY RESTORER NUCLEIC ACIDS, MARKERS, METHODS, AND ZYGOSITY ASSAYS

(71) Applicant: PIONEER HI-BRED INTERNATIONAL, INC., JOHNSTON, IA (US)

(72) Inventors: SARAH ATWOOD, URBANDALE, IA (US); KEVIN A. FENGLER, CLIVE, IA (US); SIVA S. AMMIRAJU JETTY, JOHNSTON, IA (US); JONATHAN MYRVOLD, SACRAMENTO, CA (US)

(73) Assignee: PIONEER HI-BRED INTERNATIONAL, INC., JOHNSTON, IA (US)

(21) Appl. No.: 19/109,439

(22) PCT Filed: Aug. 30, 2023
(86) PCT No.: PCT/US2023/073142

§ 371 Date: Mar. 6, 2025

Related U.S. Application Data
(60) Provisional application No. 63/374,849, filed on Sep. 7, 2022.

(51) Int. Cl. A01H 1/04; A01H 1/02; C12Q 1/6809; C12Q 1/686; C12Q 1/6895

(52) U.S. Cl. CPC A01H 1/045; A01H 1/023; C12Q 1/6809; C12Q 1/686; C12Q 1/6895; C12Q 2600/13

(57) ABSTRACT

This disclosure concerns methods and compositions for identifying plants that have an Ogura CMS restorer of fertility phenotype. Some aspects concern a zygosity assay to identify, select, and/or construct Ogura CMS restorer of fertility plants and germplasm. Particular aspects concern a zygosity assay to maintain an Ogura fertility restoration system for hybrid canola production. This disclosure also concerns plants comprising an Ogura fertility restoration system for hybrid canola production generated by methods utilizing the zygosity assay described herein.

### REFERENCE TO SEQUENCE LISTING SUBMITTED ELECTRONICALLY

The official copy of the sequence listing is submitted electronically via Patent Center as an XML formatted sequence listing with a file named 8927.xml created on Sep. 1, 2022 and having a size of 8,730 bytes and is filed concurrently with the specification. The sequence listing comprised in this XML, formatted document is part of the specification and is herein incorporated by reference in its entirety.

### FIELD OF THE INVENTION

The subject disclosure relates to plant fertility genes, methods, markers, and polynucleotides that relate to a novel truncated version of the Ogura nuclear fertility restorer segment (truncated Rf segment). The disclosed genes, methods, markers, and polynucleotides can be used to identify plant materials that containing the truncated Rf segment, to maintain a cytoplasmic male sterility (CMS) breeding system, and for canola hybrid seed production.

### BACKGROUND

Hybrid seed production in canola (Brassica napus) relies on an Ogura cytoplasmic male sterility system (CMS) introduced from radish (Raphanus sativus). This system, originally developed at INRA, France, has two components: a mitochondrial mutation that confers male sterility, and a nuclear restorer gene (Rfo) that restores male fertility even in the presence of the mitochondrial mutation. Hybrids are produced by crossing a male line (R line) that contains both Ogura nuclear restorer and sterile cytoplasm, with a female line (A line) that is male sterile and contains only the sterile Ogura cytoplasm. Successful production of high quality “pure” certified inbred and hybrid seed using CMS systems are typically challenged by two main contamination threats: adventitious presence due to physical admixture of seed (e.g., R line seed in A line seed) or ‘bee’ mediated cross pollen hybridization in seed production fields (e.g., R line hybridized with wild type B line pollen). In addition to the contamination threat, another major challenge exists during development of male inbreds, which is fixation of the Rf segment. A low-level presence of heterozygous Rf state can result in segregation into homozygous Rf and homozygous wild types during the seed increase process. Because all males, by design, have also sterile Ogura cytoplasm in canola breeding programs, those plants without Rf become sterile, leading to seed discards. This is a frequently encountered problem when males are developed using, a) pedigree selection method (instead of doubled haploid), b) in new trait development process where males are frequently incorporated with novel variation from other canola germplasm without Rf (a vast majority of all canola traits are developed this way), and c) more rarely, in trait integration between B×R line crosses. Accidental transfer of male seed with low level Rf heterozygotes can result in large scale segregation and sterility in fields aimed at inbred seed increase in seed production setting. All the above issues could result in large scale seed lot discards, delayed product launches, and compromised operational excellence leading to increased cost of goods.

These risks could be mitigated by applying stringent quality control measures via application of low cost, high-throughput, co-dominant DNA markers throughout the seed production process. Available versions of the Ogura radish restorer gene (Rfo) containing segments have been cloned and published. Each is a copy number variant (CNV) and is not found in wildtype canola. Therefore, it was not possible to develop a functional co-dominant SNP marker. In addition, the radish DNA sequence is very divergent relative to canola restorer orthologous region. This has prevented successful sequence alignments, identification and development of flanking, tightly linked co-dominant SNP assays within the radish Rf introgression.

The original Ogura restorer introgression segment from radish is very large in length, equivalent to nearly one-third of the N19 chromosome. Although, multiple versions of the Rf segment have been developed, many are known to retain undesirable oil traits and/or other composition traits. Therefore, there is a desire for alternative version of the Rf segments for use in CMS systems.

### SUMMARY OF THE DISCLOSURE

The compositions and methods disclosed herein are based, at least in part, on the discovery and development of an improved version of the Ogura Rf segment that has been shortened using gamma ray mutagenesis. The truncated Ogura Rf segment (truncated Rf segment) is located at the telomeric end of Brassica napus chromosome N19 and replaces a portion of the B. napus endogenous genomic sequence. As compared to B. napus plants homozygous for known Ogura segments that provide cytoplasmic male sterility, the truncated Rf segment disclosed herein confers important advantages. Due to their size, conventional Ogura segments have been associated with linkage drag and undesirable compositional profiles in B. napus. By contrast, the disclosed truncated Rf segment reduces linkage drag and enables lower glucosinolate content, including a glucosinolate content of less than 30 μmol/gram of seed.

Thus, provided herein are methods, assay, and reagents for detecting and determining zygosity of the truncated Rf segment in a B. napus plant, cell, or germplasm. These can be used to specifically detect the presence or absence of the truncated Rf segment and to distinguish between the truncated Rf segment and wildtype B. napus N19 genomic sequence at the same N19 locus. The present disclosure provides sequences, primers and/or probes which are useful for detecting the truncated Rf segment that also detects B. napus endogenous genomic DNA from chromosome N19.

In one aspect, disclosed herein, is a method of identifying a B. napus plant, cell, or germplasm thereof comprising a truncated Ogura Rf segment (truncated Rf segment) which includes (a) obtaining a sample comprising nucleic acid from the B. napus plant, cell, or germplasm; and (b) screening the sample for the truncated Ogura Rf segment on chromosome N19, wherein the segment is less than 5,000 kb, e.g., about 4,900 kb in length, and wherein the presence of the segment contributes to a fertility restorer phenotype in B. napus. The method can further include (c) selecting the B. napus plant, cell, or germplasm from which the sample was obtained, and which comprises the truncated Rf segment. Optionally, this method further comprises screening for the presence of a displaced endogenous B. napus genomic segment on the telomeric end of chromosome N19, wherein the genomic segment is about 2,100 kb, and wherein the presence of the genomic segment contributes to a wildtype phenotype in B. napus and indicates the absence of the truncated Rf segment on chromosome N19. Screening for both the truncated Rf segment and the displaced endogenous B. napus genomic segment at the same locus can be used to determine the zygosity of the truncated Rf segment allele.

Screening samples for the presence or absence or zygosity of the truncated Ogura Rf segment (and optionally, the displaced N19 genomic segment) can be done by any method suitable for detecting the truncated Rf segment. Thus, the method for screening a sample for the truncated Ogura Rf segment on chromosome N19 can include screening the sample to detect the presence of or absence of SEQ ID NO:2, which is the truncated Rf segment, in the sample. Screening a sample for zygosity of the truncated Rf segment can include analyzing the sample by sequencing or array hybridization to detect the presence of or absence of both (i) SEQ ID NO:2 and (ii) the sequence of SEQ ID NO:1, which is wildtype (non-Rfo) sequence at the same locus, thereby determining the zygosity of the truncated Rf segment. The screening for the presence of or absence of SEQ ID NO:2 and SEQ ID NO: 1 can be done, for example, by nucleotide sequencing or array hybridization.

In another aspect, allele-specific marker detection, such as a TaqMan® assay or competitive allele-specific PCR, can be used for zygosity analysis, including for high-throughput analysis. These methods can detect and quantify the zygosity of the truncated Rf segment in B. napus plants in a single reaction. For example, the reaction can include the use of fluorescence-based PCR method with the plate being subsequently read on a plate reader. The subject assay offers a reliable, consistent, and cost-effective method for determining the zygosity state of the truncated Rf segment in a B. napus plant, cell, or germplasm.

In some examples, a method for identifying a B. napus plant, cell, or germplasm thereof comprising a truncated Rf segment is a PCR method that includes the steps of (a) contacting the isolated nucleic acid sample with a restorer forward primer and restorer reverse primer to selectively produce an amplicon that includes sequence from the truncated Rf segment; (b) optionally, contacting the isolated nucleic acid sample with a wildtype forward primer and wildtype reverse primer to selectively produce a second amplicon that includes sequence from the displaced wildtype N19 genomic segment; (c) contacting the amplicon with a restorer probe to detect amplified genomic sequence from the truncated Rf segment; and (d) optionally, contacting the second amplicon with a wildtype probe to detect amplified genomic sequence from the displaced wildtype N19 genomic segment. For example, the truncated Rf segment restorer forward and reverse primers can comprise the sequences of SEQ ID NO:4 and SEQ ID NO: 5, the truncated Rf segment restorer probe can comprise SEQ ID NO:3, the wildtype forward and revers primers can comprise SEQ ID NO:7 and SEQ ID NO:8, and the wildtype probe comprises SEQ ID NO:6. The primers or probes used in such a PCR assay can be labeled, e.g., with a radioactive or fluorescent label for detection of amplified product. When labeled primers or probes are used, preferably the truncated Rf segment primer differs from the wildtype primer label.

The methods, assay, and molecular marker can be used with a Brassica crop plant. As used herein, Brassica preferably refers to Brassica napus, Brassica juncea, Brassica carinata, Brassica rapa, or Brassica oleracea.

A method of introgressing a fertility restorer trait into a B. napus plant is provided herein. The method includes the steps of (a) crossing a first parent B. napus plant comprising a truncated Ogura Rf segment with a second parent B. napus plant that does not have the segment to produce hybrid progeny plants; (b) obtaining a nucleic acid sample from one or more hybrid progeny plants; and (c) screening the sample for the presence or absence of the truncated Rf segment in accordance with any suitable method disclosed herein (e.g., by sequencing, allele-specific amplification, TaqMan® assay, gel-based assay, etc.) (d) selecting the one or more progeny plants based on their samples having the truncated Rf segment. In one example, the method of introgression can further include (e) crossing the one or more selected progeny plants with the first or second parent B. napus plant (the recurrent parent plant) to produce backcross progeny plants; (e) obtaining a nucleic acid sample from one or more backcross progeny plants; (f) selecting the one or more backcross progeny plants having the truncated Rf segment to produce another generation of backcross progeny plants; and (g) repeating steps (d), (e), and (f) three or more times to produce backcross progeny plants that comprise the truncated Ogura Rf segment and the agronomic characteristics of the recurrent parent plant when grown in the same environmental conditions. Also disclosed herein is the introgressed B. napus plant produced by the described method.

A method for restoring male fertility in B. napus is disclosed herein. The method includes the steps of (a) crossing a male restorer line that contains both the truncated Rf segment and a female line that is male sterile and does not contain the truncated Rf segment to generate F₁ B. napus plants; (b) screening sample from the F₁ B. napus plants for the presence or absence of the truncated Rf segment in accordance with any suitable method disclosed herein (e.g., by sequencing, allele-specific amplification, TaqMan® assay, gel-based assay, etc.), (c) selecting the F₁ B. napus plants having the truncated Rf segment; and (c) propagating the identified F₁ B. napus plant, thereby restoring male fertility in the B. napus plant.

In one aspect, the PCR assay method disclosed herein is used for determining zygosity of a truncated Ogura Rf segment in a B. napus plant, cell or germplasm, wherein the method includes the steps of (a) performing a first PCR assay using a first probe, a first forward primer, and a first reverse primer on a polynucleotide from a B. napus plant sample, wherein the first probe is SEQ ID NO:3: (b) performing a second PCR assay using a second probe, a second forward primer, and a second reverse primer on the polynucleotide sample, wherein the second probe is SEQ ID NO:6; (c) quantifying the first probe and the second probe; and (d) comparing the quantified first probe and the quantified second probe of the first PCR assay and the second PCR assay to determine the zygosity. In an aspect, the first probe detects the presence of the truncated Ogura Rf segment, and the second probe detects the displaced wildtype N19 genomic segment. In an aspect, the probes are detectably labeled. In a further aspect, the first and second probes are labeled with both a fluorescent dye and quencher. In another aspect, the first forward and reverse primers comprise SEQ ID NO:4 and SEQ ID NO:5, and the second forward and reverse primers comprise SEQ ID NO:7 and SEQ ID NO:8. The first primers and probes are specific for the Ogura Rf segment in a B. napus plant. The second primers and probes are specific for endogenous B. napus N19 sequence in a B. napus plant. In some examples, the PCR assay method comprises a TaqMan® assay, such as a TaqMan® zygosity assay.

In yet another aspect, the truncated Ogura Rf segment can be characterized using reference genomes for two R-lines that contain reduced Ogura Rf segment, and for two B-lines. By comparatively analyzing orthologous N19 chromosomal region containing Ogura Rf introgression between these R and B lines, along with publicly available diploid radish orthologous chromosome 9 sequence, the attributes of the truncated Rf introgression are precisely characterized, including identification of introgression break points at base pair level. This characterization of the truncated Ogura Rf segment allows for the development of a TaqMan® PCR assay that uses oligonucleotides specific to the Ogura Rf segment and oligonucleotides corresponding to endogenous B. napus genomic DNA in a single multiplex reaction.

In one example, provided herein is a TaqMan® marker zygosity assay. The Ogura Rf segment is amplified with oligonucleotide primers SEQ ID NOs: 4 and 5 and detected with oligonucleotide probe SEQ ID NO: 3. In a further aspect, the displaced endogenous B. napus genomic sequence is amplified with oligonucleotide primers SEQ ID NOs: 7 and 8 and detected with oligonucleotide probe SEQ ID NO: 6. In one result of the assay, primers SEQ ID NOs: 4 and 5 and probe SEQ ID NO: 3 detect exogenous radish sequence comprising the Ogura Rf segment and do not detect wildtype endogenous B. napus sequence in a genomic region displaced by an exogenous radish sequence comprising the Ogura Rf segment. In another result, primers SEQ ID NOs: 7 and 8 and probe SEQ ID NO: 6 detect endogenous B. napus sequence in a genomic region displaced by an exogenous radish sequence comprising the truncated Ogura Rf segment and do not detect the exogenous radish sequence comprising the truncated Ogura Rf segment. The endogenous B. napus sequence is about 2,100 kb long and is at the telomeric end of chromosome N19.

The disclosed TaqMan® PCR based zygosity assay can be a multiplex assay that uses relative intensity of fluorescence wavelengths that are specific for each of the Ogura Rf segment and the endogenous B. napus genomic sequence. In an aspect, the subject assay is used to identify lines of B. napus that possess restored male fertility and lines of B. napus that are male sterile.

In some aspects, each of the disclosed methods for detecting the truncated Ogura Rf segment may be used to maintain the Ogura cytoplasmic male sterility system. Thus, the methods disclosed herein can be used to confirm: (a) female inbred purity by demonstrating the absence of the truncated Ogura Rf segment in A lines; (b) maintainer line purity by demonstrating the absence of the truncated Ogura Rf segment in B lines; (c) male inbred purity and uniformity or fixity of the truncated Ogura Rf segment by demonstrating homozygous state of the truncated Ogura Rf segment in the male inbred lines; and (d) hybrid purity and uniformity by demonstrating the heterozygous state of the truncated Ogura Rf segment in the hybrids.

Methods and compositions disclosed herein are used to develop a restorer B. napus plant, wherein the plant comprises the truncated Ogura Rf segment that is about 4,900 kb in length and does not comprise the wildtype N19 genomic segment that is about 2,100 kb in length.

### BRIEF DESCRIPTION OF THE DRAWINGS AND THE SEQUENCE LISTING

The disclosure can be more fully understood from the following detailed description and the accompanying drawings and Sequence Listing, which form a part of this application.

FIG. 1 depicts alignment of radish segment replacement in R-line G00555MC with B-line NS1822BC on N19.

FIG. 2 depicts the schematic for TaqMan® assay N101TA9-00-Q001.

The sequence descriptions and sequence listing attached hereto comply with the rules governing nucleotide and amino acid sequence disclosures in patent applications as set forth in 37 C.F.R. §§ 1.831-1.835. Sequences are listed in Table 1.

TABLE 1

| SEQ ID NO: | Name | Description |
|---|---|---|
| 1 | rf_n19_wt_1910704_1910716 | Wildtype (Non-Rfo) target sequence located on |
| | | NS1822BC.GOLD.CHROMOSOMES_v2 at N19: |
| | | 1910704-1910716 bp. |
| 2 | rf_n19_mut_01 | Mutant (Restorer) target sequence located on |
| | | Canola4_G00555MC.CHROMOSOMES at |
| | | N19: 4939371-4939478 bp. |
| 3 | N101T5R-001-X004 | Restorer VIC Probe |
| 4 | N101T5R-F002 | Restorer Forward Primer 1 |
| 5 | N101T5R-R003 | Restorer Reverse Primer 1 |
| 6 | N101T7A-001-X001 | Wildtype FAM Probe |
| 7 | N101T7A-F001 | Wildtype Forward Primer 2 |
| 8 | N101T7A-R001 | Wildtype Reverse Primer 2 |

### DETAILED DESCRIPTION

The disclosure of all patents, patent applications, and publications cited herein are incorporated by reference in their entirety.

Terms used in the claims and specification are defined as set forth below unless otherwise specified. It must be noted that, as used in the specification and the appended claims, the singular forms “a,” “an” and “the” include plural referents unless the context clearly dictates otherwise.

Numeric ranges are inclusive of the numbers defining the range and include each integer and non-integer fraction within the defined range. Unless defined otherwise, all technical and scientific terms used herein have the same meaning as commonly understood by one of ordinary skill in the art.

### Definitions

In order to facilitate review of the various embodiments described in this disclosure, the following explanation of specific terms is provided:

The term “allele” refers to an alternative form of a gene, whereby two genes can differ in DNA sequences. Such differences may result from at least one mutation (e.g., deletion, insertion, and/or substitution) in the nucleic acid sequence. Alleles may result in modified mRNAs or polypeptides whose structure or function may or may not be modified. Any given gene may have none, one, or many allelic forms. Each of these types of changes may occur alone, or in combination with the others, one or more times in a given sequence.

An “amplicon” is amplified nucleic acid, e.g., a nucleic acid that is produced by amplifying a template nucleic acid by any available amplification method (e.g., PCR, LCR, transcription, or the like).

The term “backcrossing” refers to methods used to introduce a nucleic acid sequence into a plant. The backcrossing technique has been widely used for decades to introduce new traits into plants (Jensen, N., Ed. Plant Breeding Methodology, John Wiley & Sons, Inc., 1988). In a typical backcross protocol, the original variety of interest (recurrent parent) is crossed to a second variety (non-recurrent parent) that carries a gene of interest to be transferred. The resulting progeny from this cross are then crossed again to the recurrent parent, and the process is repeated until a plant is obtained wherein essentially all of the desired morphological and physiological characteristics of the recurrent plant are recovered in the converted plant, in addition to the transferred gene or locus from the nonrecurrent parent.

“Brassica” refers to any one of Brassica napus (AACC, 2n=38), Brassica juncea (AABB, 2n=36), Brassica carinata (BBCC, 2n=34), Brassica rapa (syn. B. campestris) (AA, 2n=20), Brassica oleracea (CC, 2n=18) or Brassica nigra (BB, 2n=16). The term “canola” refers to Brassica napus or rapeseed and includes all plant varieties that can be bred with canola, including other Brassica species.

The term “elite” line in the context of canola hybrid, canola inbred, or another plant variety generally refers to any line that has resulted from breeding and selection for superior agronomic performance, for example and without limitation: improved yield of at least one plant commodity product; maturity; disease resistance; and standability. An elite plant is any plant from an elite line.

The term “endogenous” or “native” nucleic acid is a nucleic acid (e.g., a gene) that does not contain a nucleic acid element other than those normally present in the chromosome or other genetic material on which the nucleic acid is normally found in nature. An endogenous gene transcript is encoded by a nucleotide sequence at its natural chromosomal locus and is not artificially supplied to the cell. In some aspects, an endogenous nucleic acid of particular interest is a B. napus nucleic acid that has been replaced in the B. napus genome by an exogenous radish nucleic acid comprising the Ogura Rf segment. This endogenous nucleic acid may be reintroduced into the B. napus genome in some embodiments by crossing a B. napus plant comprising the exogenous radish nucleic acid with a plant comprising the endogenous nucleic acid, such that the endogenous nucleic acid is integrated into the B. napus genome in its normal position, replacing the exogenous radish nucleic acid.

The term “exogenous” refers to a nucleic acid that is not native to a specified system (e.g., a germplasm, variety, elite variety, and/or plant) with respect to nucleotide sequence and/or genomic location for a polynucleotide, and with respect to amino acid sequence and/or cellular localization for a polypeptide. In aspects, exogenous or heterologous polynucleotides or polypeptides may be nucleic acids that have been artificially supplied to a biological system (e.g., a plant cell, a plant gene, a particular plant species or variety, and/or a plant chromosome) and are not native to that particular biological system. Thus, the designation of a nucleic acid as “exogenous” may indicate that the nucleic acid originated from a source other than a naturally-occurring source, or it may indicate that the nucleic acid has a non-natural configuration, genetic location, or arrangement of elements. In some aspects, an exogenous nucleic acid of particular interest is a radish nucleic acid integrated in the B. napus genome, wherein the radish nucleic acid comprises the Ogura Rf segment.

The term “gene” (or “genetic element”) may refer to a heritable genomic DNA sequence with functional significance, for example, an exogenous Ogura Rf gene of radish origin that has been incorporated into a canola genome. The term “gene” may also be used to refer to, for example and without limitation, a cDNA and/or an mRNA encoded by a heritable genomic DNA sequence.

A “genetic map” is a description of genetic linkage relationships among loci on one or more chromosomes (or linkage groups) within a given species, as may be determined by analysis of a mapping population. In some examples, a genetic map may be depicted in a diagrammatic or tabular form. The term “genetic mapping” may refer to the process of defining the linkage relationships of loci through the use of genetic markers, mapping populations segregating for the markers, and standard genetic principles of recombination frequency. A “genetic map location” refers to a location on a genetic map (relative to surrounding genetic markers on the same linkage group or chromosome) where a particular marker can be found within a given species. In contrast, a “physical map of the genome” refers to absolute distances (for example, measured in base pairs or isolated and overlapping contiguous genetic fragments) between markers within a given species. A physical map of the genome does not necessarily reflect the actual recombination frequencies observed in a test cross of a species between different points on the physical map.

“Genome” as it applies to plant cells encompasses not only chromosomal DNA found within the nucleus, but organelle DNA found within subcellular components (e.g., mitochondrial, plastid) of the cell.

As used herein, the term “genotype” refers to the genetic constitution of an individual (or group of individuals) at one or more particular loci. The genotype of an individual or group of individuals is defined and described by the allele forms at the one or more loci that the individual has inherited from its parents. The term genotype may also be used to refer to an individual's genetic constitution at a single locus, at multiple loci, or at all the loci in its genome. A “haplotype” is the genotype of an individual at a plurality of genetic loci. In some examples, the genetic loci described by a haplotype may be physically and genetically linked; i.e., the loci may be positioned on the same chromosome segment and may be inherited together.

The term “germplasm” refers to genetic material of or from an individual plant or group of plants (e.g., a plant line, variety, and family), and a clone derived from a plant or group of plants. A germplasm may be part of an organism or cell, or it may be separate (e.g., isolated) from the organism or cell. In general, germplasm provides genetic material with a specific molecular makeup that is the basis for hereditary qualities of the plant. As used herein, “germplasm” refers to cells of a specific plant; seed; tissue of the specific plant (e.g., tissue from which new plants may be grown); and non-seed parts of the specific plant (e.g., leaf, stem, pollen, and cells). As used herein, the term “germplasm” is synonymous with “genetic material,” and it may be used to refer to seed (or other plant material) from which a plant may be propagated A “germplasm bank” may refer to an organized collection of different seed or other genetic material (wherein each genotype is uniquely identified) from which a known cultivar may be cultivated, and from which a new cultivar may be generated. In embodiments, a germplasm utilized in a method or plant as described herein is from a canola inbred or hybrid. In particular examples, a germplasm is seed of the canola inbred or hybrid. In particular examples, a germplasm is a nucleic acid sample from the canola inbred or hybrid.

The term “introgression” refers to the movement of a gene or genes through sexual crossing, usually by pollen, from a plant which is intended to be the donor for the formation of seed.

The term “locus” refers to a position on the genome that corresponds to a measurable characteristic (e.g., a trait). An “Ogura Rf segment,” “Rf segment,” and “Ogura Rf fragment” refers to a Raphanus sativus DNA fragment including a fertility Rfo gene for Ogura cytoplasmic male sterility. A “truncated Ogura Rf segment,” “truncated Rf segment,” and “truncated Ogura Rf segment,” refers to the specific 4,900 kb R. sativus DNA fragment including a fertility Rfo gene for Ogura cytoplasmic male sterility which has been introgressed into B. napus as described herein.

The terms “marker” and “molecular marker” refer to a nucleic acid or encoded product thereof (e.g., a protein) used as a point of reference when identifying a linked locus. Thus, a marker may refer to a gene or nucleotide sequence that can be used to identify plants having a particular allele, e.g., Rf. A marker may be described as a variation at a given genomic locus. A genetic marker may be a short DNA sequence, such as a sequence surrounding a single base-pair change (single nucleotide polymorphism, or “SNP”), or a long one, for example, a sequence within an insertion or deletion. The term “marker allele” refers to the version of the marker that is present in a particular plant. The term marker as used herein may refer to a fragment of radish chromosomal DNA (for example, as defined by SEQ ID NOs: 3, 4, or 5) or to a fragment of B. napus chromosomal DNA (for example, as defined by SEQ ID NOs: 6, 7, or 8).

DNA may develop and accumulate polymorphism for any of a variety of reasons, and therefore may be variable between individuals of the same species. The genomic variability can be of any origin, for example, the variability may be due to DNA insertions, deletions, duplications, repetitive DNA elements, point mutations, recombination events, and the presence and sequence of transposable elements. Such regions may contain useful molecular genetic markers. In general, any differentially inherited polymorphic trait (including nucleic acid polymorphisms) that segregates among progeny is a potential marker.

A genetic marker may be a short DNA sequence, such as a sequence surrounding a single base-pair change (single nucleotide polymorphism, or “SNP”), or a long one, for example, a microsatellite/simple sequence repeat (“SSR”). A “marker allele” or “marker allele form” refers to the version of the marker that is present in a particular individual. The term “marker” as used herein may refer to a cloned segment of chromosomal DNA and may also or alternatively refer to a DNA molecule that is complementary to a cloned segment of chromosomal DNA. The term also refers to nucleic acid sequences complementary to genomic marker sequences, such as nucleic acid primers and probes.

A marker may be described, for example, as a specific polymorphic genetic element at a specific location in the genetic map of an organism. A genetic map may be a graphical representation of a genome (or a portion of a genome, such as a single chromosome) where the distances between landmarks on the chromosome are measured by the recombination frequencies between the landmarks. A genetic landmark can be any of a variety of known polymorphic markers, for example and without limitation: simple sequence repeat (SSR) markers; restriction fragment length polymorphism (RFLP) markers; and single nucleotide polymorphism (SNP) markers. As one example, SSR markers can be derived from genomic or expressed nucleic acids (e.g., expressed sequence tags (ESTs)).

Additional markers include, for example and without limitation, ESTs; amplified fragment length polymorphisms (AFLPs) (Vos et al., 1995, Nucl. Acids Res. 23:4407; Becker et al., 1995, Mol. Gen. Genet. 249:65; Meksem et al., 1995, Mol. Gen. Genet. 249:74); randomly amplified polymorphic DNA (RAPD); and isozyme markers. Isozyme markers may be employed as genetic markers, for example, to track isozyme markers or other types of markers that are linked to a particular first marker. Isozymes are multiple forms of enzymes that differ from one another with respect to amino acid sequence (and therefore with respect to their encoding nucleic acid sequences). Some isozymes are multimeric enzymes containing slightly different subunits. Other isozymes are either multimeric or monomeric but have been cleaved from a pro-enzyme at different sites in the pro-enzyme amino acid sequence. Isozymes may be characterized and analyzed at the protein level or at the nucleic acid level. Thus, any of the nucleic acid-based methods described herein can be used to analyze isozyme markers in particular examples.

Accordingly, genetic marker alleles that are polymorphic in a population can be detected and distinguished by one or more analytic methods such as, PCR-based allele-specific amplification methods, RFLP analysis, AFLP analysis, isozyme marker analysis, SNP analysis, SSR analysis, allele specific hybridization (ASH) analysis, detection of amplified variable sequences of the plant genome, detection of self-sustained sequence replication, detection of simple sequence repeats (SSRs), randomly amplified polymorphic DNA (RAPD) analysis. Thus, in certain examples of the invention, such known methods can be used to detect the Ogura Rf segment and flanking sequence(s) as well as the molecular markers for detecting the presence or absence of the Ogura Rf segment which are disclosed herein. See, e.g., Table 1 herein.

Numerous statistical methods for determining whether markers are genetically linked to a locus or gene (or to another marker) are known to those of skill in the art and include, for example and without limitation, standard linear models (e.g., ANOVA or regression mapping; Haley and Knott, 1992, Heredity 69:315); and maximum likelihood methods (e.g., expectation-maximization algorithms; Lander and Botstein, 1989, Genetics 121:185-99; Jansen, 1992, Theor. Appl. Genet. 85:252-60; Jansen, 1993, Biometrics 49:227-31; Jansen, 1994, “Mapping of quantitative trait loci by using genetic markers: an overview of biometrical models,” In J. W. van Ooijen and J. Jansen (eds.), Biometrics in Plant breeding: applications of molecular markers, pp. 116-24 (CPRO-DLO Netherlands); Jansen, 1996, Genetics 142:305-11; and Jansen and Stam, 1994, Genetics 136:1447-55).

Exemplary statistical methods include single point marker analysis; interval mapping (Lander and Botstein, 1989, Genetics 121:185); composite interval mapping; penalized regression analysis; complex pedigree analysis; MCMC analysis; MQM analysis (Jansen, 1994, Genetics 138:871); HAPLO-IM+ analysis, HAPLO-MQM analysis, and HAPLO-MQM+ analysis; Bayesian MCMC; ridge regression; identity-by-descent analysis; and Haseman-Elston regression, any of which are suitable in the context of particular embodiments of the invention. Alternative statistical methods applicable to complex breeding populations that may be used to identify and localize loci or QTLs are described in U.S. Pat. No. 6,399,855 and PCT International Patent Publication No. WO0149104 A2. All of these approaches are computationally intensive and are usually performed with the assistance of a computer-based system comprising specialized software. Appropriate statistical packages are available from a variety of public and commercial sources, and are known to those of skill in the art.

The term “marker-assisted selection (MAS)” is the process of indirect selection of agriculturally important traits using morphological, biochemical, or DNA markers in crop breeding. Breeding with the use of markers is termed “marker-assisted breeding (MAB)” and it provides a time- and cost-efficient process for improvement of plant varieties.

Molecular marker technologies generally increase the efficiency of plant breeding through MAS. A molecular marker allele that demonstrates linkage disequilibrium with a desired phenotypic trait provides a useful tool for the selection of the desired trait in a plant population. The key components to the implementation of an MAS approach are the creation of a dense (information rich) genetic map of molecular markers in the plant germplasm; the detection of at least one locus, gene, or QTL based on statistical associations between marker and phenotypic variability; the definition of a set of particular useful marker alleles based on the results of the mapping analysis; and the use and/or extrapolation of this information to the current set of breeding germplasm to enable marker-based selection decisions to be made.

The closer a particular marker is to a gene that encodes a polypeptide that contributes to a particular phenotype (whether measured in terms of genetic or physical distance), the more tightly linked is the particular marker to the phenotype. In view of the foregoing, it will be appreciated that the closer (whether measured in terms of genetic or physical distance) that a marker is linked to a particular gene, the more likely the marker is to segregate with that gene (e.g., Ogura Rf segment disclosed herein) and its associated phenotype (e.g., the restorer function of the Ogura Rf segment disclosed herein). Thus, the extremely tightly linked genetic markers of the Ogura Rf segment disclosed herein can be used in MAS programs to identity canola varieties that have or can generate progeny that have a restorer phenotype (when compared to parental varieties and/or otherwise isogenic plants lacking the Ogura Rf segment), to identify individual canola plants comprising this restorer trait, and to breed this trait into other canola varieties to provide the restorer trait. In some aspects, marker assisted breeding and/or phenotypic selection can be used either simultaneously or sequentially to select restorer canola plants.

As used herein, a “nucleic acid molecule” is a polymeric form of nucleotides, which can include both sense and anti-sense strands of RNA, cDNA, genomic DNA, and synthetic forms and mixed polymers of the above. A nucleotide refers to a ribonucleotide, deoxynucleotide, or a modified form of either type of nucleotide. A “nucleic acid molecule” as used herein is synonymous with “nucleic acid”, “nucleotide sequence”, “nucleic acid sequence”, and “polynucleotide.” The term includes single- and double-stranded forms of DNA. A nucleic acid molecule can include either or both naturally occurring and modified nucleotides linked together by naturally occurring and/or non-naturally occurring nucleotide linkages.

Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications, such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., peptides), intercalators (e.g., acridine, psoralen, etc.), chelators, alkylators, and modified linkages (e.g., alpha anomeric nucleic acids, etc.). The term “nucleic acid molecule” also includes any topological conformation, including single-stranded, double-stranded, partially duplexed, triplexed, hairpinned, circular, and padlocked conformations.

The term “oligonucleotide” refers to a single-stranded nucleic acid including at least between two and about 100 natural or modified nucleotides or a mixture thereof. The oligonucleotide can be derived from a natural nucleic acid or produced by chemical or enzymatic synthesis.

As used herein, “phenotype” means the detectable characteristics (e.g., restorer function) of a cell or organism which can be influenced by genotype.

The term “plant” includes reference to whole plants, plant parts, seeds, plant cells, and progeny of same. Plant cells include, without limitation, cells from seeds, suspension cultures, embryos, meristematic regions, callus tissue, leaves, roots, shoots, gametophytes, sporophytes, pollen, and microspores. “Progeny” comprises any subsequent generation of a plant.

As used herein, the term “plant material” refers to any processed or unprocessed material derived, in whole or in part, from a plant. For example, and without limitation, a plant material may be a plant part, a seed, a fruit, a leaf, a root, a plant tissue, a plant tissue culture, a plant explant, or a plant cell.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label.

“Polypeptide” refers to a peptide or protein containing two or more amino acids linked by peptide bonds, and includes peptides, oligomers, proteins, and the like. Polypeptides can contain natural, modified, or synthetic amino acids. Polypeptides can also be modified naturally, such as by post-translational processing, or chemically, such as amidation, acylation, cross-linking, and the like.

“Polymerase chain reaction” or “PCR” refers to a procedure or technique in which minute amounts of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et ah, Cold Spring Harbor Symp. Quant. Biol., 51:263 (1987); Erlich, ed., PCR Technology, (Stockton Press, NY, 1989).

The term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. A primer is preferably a single strand sequence, such that amplification efficiency is optimized, but double stranded sequences can be utilized.

The term “probe” refers to an oligonucleotide that hybridizes to a target sequence. In the TaqMan® or TaqMan®-style assay procedure, the probe hybridizes to a portion of the target situated between the annealing site of the two primers. A probe can further include a detectable label, e.g., a fluorophore (Texas-Red®, Fluorescein isothiocyanate, etc.,). The detectable label can be covalently attached directly to the probe oligonucleotide, e.g., located at the probe's 5′ end or at the probe's 3′ end. A probe including a fluorophore may also further include a quencher, e.g., Black Hole Quencher™, Iowa Black™, etc. A probe includes about eight nucleotides, about ten nucleotides, about fifteen nucleotides, about twenty nucleotides, about thirty nucleotides, about forty nucleotides, or about fifty nucleotides. In some embodiments, a probe includes from about eight nucleotides to about fifteen nucleotides.

The term “quenching” refers to a decrease in fluorescence of a fluorescent detectable label caused by energy transfer associated with a quencher moiety, regardless of the mechanism.

The term “reaction mixture” or “PCR reaction mixture” or “master mix” or “master mixture” refers to an aqueous solution of constituents in a PCR or RT-PCR reaction that can be constant across different reactions. An exemplary RT-PCR reaction mixture includes buffer, a mixture of deoxyribonucleoside triphosphates, reverse transcriptase, primers, probes, and DNA polymerase. Generally, template RNA or DNA is the variable in a PCR or RT-PCR reaction.

The term “sample” refers to a part of any plant species, but preferably is from canola (Brassica napus). Such can be at the macro or micro level, wherein polynucleotides and/or polypeptides can be extracted.

The terms “specifically hybridizable” and “specifically complementary” are terms that indicate a sufficient degree of complementarity such that stable and specific binding occurs between the nucleic acid molecule and the DNA target. A nucleic acid molecule need not be 100% complementary to its target sequence to be specifically hybridizable. A nucleic acid molecule is specifically hybridizable when there is a sufficient degree of complementarity to avoid non-specific binding of the nucleic acid to non-target sequences under conditions where specific binding is desired, for example, under stringent hybridization conditions.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na+ and/or MgA concentration) of the hybridization buffer will determine the stringency of hybridization, though wash times also influence stringency.

Calculations regarding hybridization conditions required for attaining particular degrees of stringency are known to those of ordinary skill in the art, and are discussed, for example, in Sambrook et al. (ed.) Molecular Cloning: A Laboratory Manual, 2nd ed., vol. 1-3, Cold Spring Harbor Laboratory Press, Cold Spring Harbor, NY, 1989, chapters 9 and 11; and Hames and Higgins (eds.) Nucleic Acid Hybridization, IRL Press, Oxford, 1985. Further detailed instruction and guidance with regard to the hybridization of nucleic acids may be found, for example, in Tijssen, “Overview of principles of hybridization and the strategy of nucleic acid probe assays,” in Laboratory Techniques in Biochemistry and Molecular Biolofiy-Hybridization with Nucleic Acid Probes, Part I, Chapter 2, Elsevier, NY, 1993; and Ausubel et ah, Eds., Current Protocols in Molecular Biology, Chapter 2, Greene Publishing and Wiley-Interscience, NY, 1995.

The term “zygosity” refers to the similarity of alleles for a gene or trait in an organism (e.g., a plant). If both alleles are the same, the organism is homozygous for the allele. If the two alleles are different, the organism is heterozygous for the gene or trait. If one allele is not present, the organism is hemizygous. If both alleles are not present, the organism is nullizygous. The term “label” when used herein refers to a detectable compound or composition that is conjugated directly or indirectly to a probe to generate a “labeled” probe. The label may be detectable by itself (e.g. radioisotope labels or fluorescent labels) or, in the case of an enzymatic label, may catalyze chemical alteration of a substrate compound or composition that is detectable (e.g., avidin-biotin).

### Ogura Cytoplasmic Male Sterility System

In developing improved new Brassica varieties, breeders use self-incompatible (SI), cytoplasmic male sterile (CMS) and nuclear male sterile (NMS) Brassica plants as the female parent. In using these plants, breeders are attempting to improve the efficiency of seed production and the quality of the F₁ hybrids and to reduce the breeding costs. When hybridization is conducted without using SI, CMS or NMS plants, it is more difficult to obtain and isolate the desired traits in the progeny (F₁ generation) because the parents are capable of undergoing both cross-pollination and self-pollination. If one of the parents is a SI, CMS or NMS plant that is incapable of producing pollen, only cross pollination will occur. By eliminating the pollen of one parental variety in a cross, a plant breeder is assured of obtaining hybrid seed of uniform quality, provided that the parents are of uniform quality and the breeder conducts a single cross.

Production of Brassica F₁ hybrids includes crossing a CMS Brassica female parent, with a pollen producing male Brassica parent. CMS is the maternally-inherited inability to produce functional pollen. In the absence of a fertility restorer gene, plants of a CMS inbred are male-sterile as a result of factors from the cytoplasmic (as opposed to the nuclear) genome. Therefore, the characteristic of male sterility is inherited exclusively through the female parent, since only the female provides cytoplasm to the fertilized seed. To reproduce effectively, the male parent of the F₁ hybrid must have a fertility restorer gene (Rf gene). The presence of a Rf gene means that the generation will not be completely or partially sterile, so that either self-pollination or cross pollination may occur. Self-pollination of the F₁, generation to produce several subsequent generations is important to ensure that a desired trait is heritable and stable and that a new variety has been isolated.

The Ogura cytoplasmic male sterile (CMS) system has been developed in B. napus and utilizes ogu cytoplasm and a dominant nuclear fertility restorer gene, Rfo, from radish. The Rfo gene was originally transferred from Raphanus sativus to B. napus through intergeneric hybridization, along with a large segment of linked radish material (Heyn, 1976. Cruciferae Newsl 1:15-16; Primard-Brisset et al., 2005. Theor. Appl. Genet. 111:736-746). The original radish introgression replaced a B. napus region covering around 60 cM on chromosome N19 (Delourme et al., 1998. Theor. Appl. Genet. 97:129-134). This large radish genomic fragment carried many undesirable traits, including elevated glucosinolate levels and decreased seed set. Efforts to reduce the size of the radish introgression have been made through extensive backcross and pedigree breeding and irradiation, and while the radish segment has successfully been reduced, lost segments of introgressed radish genome have not been replaced with the corresponding segments of the originally displaced N19 B. napus genome segment. Many low-glucosinolate B. napus restorer lines have been selected since 1992, and, as a result, multiple sources of the Rfo gene are available with different radish introgression lengths. Characterizations of B. napus restorers indicate that the size of the radish introgression has decreased from its original size of about 60 CM to about 41 cM and smaller (Giancola et al., 2003. Theor. Appl. Genet. 107:1442-1451; Primard-Brisset et al., 2005).

A proprietary truncated Ogura Rf restorer has been developed by eliminating radish genomic sequences from the original radish introgression, resulting in a smaller fragment, decreased glucosinolate levels, and improved seed set. The proprietary truncated Ogura Rf segment disclosed herein has about 4,900 kb of radish sequence which includes the Rfo gene and is located at the telomeric end of chromosome N19.

In an aspect, B. napus restorer plants comprising the truncated Ogura Rf segment have an exogenous radish genomic sequence of about 1 kb, 100 kb, 200 kb, 300 kb, 400 kb, 500 kb, 600 kb, 700 kb, 800 kb, 900 kb, 1,000 kb, 1,100 kb, 1,200 kb, 1,300 kb, 1,400 kb, 1,500 kb, 1,600 kb, 1,700 kb, 1,800 kb, 1,900 kb, 2,000 kb, 2,100 kb, 2,200 kb, 2,300 kb, 2,400 kb, 2,500 kb, 2,600 kb, 2,700 kb, 2,800 kb, 2,900 kb, 3,000 kb, 3,100 kb, 3,200 kb, 3,300 kb, 3,400 kb, 3,500 kb, 3,600 kb, 3,700 kb, 3,800 kb, 3,900 kb, 4,000 kb, 4,100 kb, 4,200 kb, 4,300 kb, 4,400 kb, 4,500 kb, 4,600 kb, 4,700 kb, 4,800 kb, 4,900 kb, 5,000 kb in length and longer on telomeric end of B. napus chromosome N19. In an aspect, the proprietary truncated Ogura Rf segment is 4,940,398 bp in length.

The methods and assays of the disclosure are based on the radish introgression on telomeric end of B. napus chromosome N19. In an aspect of the present invention, a TaqMan® PCR assay was developed for zygosity testing of the proprietary truncated Ogura Rf segment. The development of this assay results in a more efficient and cost-effective Ogura CMS system for hybrid B. napus seed production.

### Detection of the Truncated Ogura Rf Segment

Aspects of the invention include a marker that is linked to the Ogura fertility restorer in B. napus. Such a marker may be used, for example and without limitation, to identify B. napus plants and germplasm having an increased likelihood of comprising a restorer phenotype; to select such B. napus plants and germplasm (e.g., in a marker-assisted selection program); and to identify and select B. napus plants and germplasm that do not have an increased likelihood of comprising a restorer phenotype. Use of the methods and compositions describe herein may provide advantages to plant breeders with respect to the time, cost, and labor involved in B. napus breeding, when compared to currently available methods and compositions described in the art. For example, the marker described herein may provide superior results in marker-assisted breeding for the CMS system of B. napus, when compared to currently available markers for this purpose.

The truncated Ogura Rf segment disclosed herein can be detected using any method for detecting polymorphisms. Additionally, such methods can be used to detect a polymorphic marker that is genetically linked to the truncated Ogura Rf segment. These methods include allele-specific amplification and PCR based amplification assays such as TaqMan, rhAmp-SNP, KASP, and molecular beacons. Such an assay can include the use of one or more probes that detect the truncated Ogura Rf segment, a marker associated with the segment, or an amplicon that is selectively produced by amplification of genomic sequence comprising all or a part of the truncated Ogura Rf segment. Optionally, such an assay can further include an additional set of primers and/or one or more probes that detect the presence of a wildtype N19 genomic sequence (e.g., wildtype allele) that includes the displaced endogenous B. napus genomic segment, as disclosed herein.

Additional methods for genotyping and detecting the truncated Ogura Rf segment (or a linked marker) include but are not limited to, hybridization, primer extension, oligonucleotide ligation, nuclease cleavage, minisequencing and coded spheres. Such methods are reviewed in publications including Gut, 2001, Hum. Mutat. 17:475; Shi, 2001, Clin. Chem. 47:164; Kwok, 2000, Pharmacogenomics 1:95; Bhattramakki and Rafalski, “Discovery and application of single nucleotide polymorphism markers in plants”, in PLANT GENOTYPING: THE DNA FINGERPRINTING OF PLANTS (CABI Publishing, Wallingford 2001). A wide range of commercially available technologies utilize these and other methods to interrogate the truncated Ogura Rf segment disclosed herein (or a linked marker), including Masscode™ (Qiagen, Germantown, MD), Invader® (Hologic, Madison, WI), SnapShot® (Applied Biosystems, Foster City, CA), Taqman® (Applied Biosystems, Foster City, CA), KASP™ (LGC Biosearch Technologies, Middlesex, UK), and Infinium Bead Chip™ and GoldenGate™ allele-specific extension PCR-based assay (Illumina, San Diego, CA).

One exemplary technique useful in practicing this invention is TaqMan® (Roche Molecular Systems, Inc., Pleasanton, CA). TaqMan® PCR provides a method to detect and/or quantifying the presence of a DNA sequence. Briefly, TaqMan® PCR utilizes a FRET oligonucleotide probe which is designed to hybridize within the target sequence for detection (i.e., the target sequence is the truncated Ogura Rf segment). The FRET probe and PCR primers (i.e., the PCR primers are designed in the truncated Ogura Rf segment) are cycled in the presence of a thermostable polymerase and dNTPs. Hybridization of the FRET probe, and subsequent digestion during the PCR amplification stage due to 5′ exonuclease activity of the Taq polymerase, results in cleavage and release of the fluorescent moiety away from the quenching moiety on the FRET probe. A fluorescent signal indicates the presence of the target sequence due to successful hybridization and amplification.

Discrimination between the target sequences is achieved using FRET combined with one or two target-specific probes that hybridize to their respective sites. When each probe is hybridized to target DNA under appropriate probe design and hybridization conditions, a single-base mismatch between the probe and target DNA prevents hybridization. In this manner, only one of the probes will hybridize to a target sample that is homozygous for a target sequence (e.g., the truncated Ogura Rf segment). Samples that are heterozygous or heterogeneous for two target sequences will hybridize to both of two alternative probes.

Additionally, the method of the present invention provides a process to efficiently analyze large numbers of B. napus samples in a high-throughput manner within a commercial setting. Another advantage of the present invention is time savings. The subject TaqMan® assay for B. napus zygosity and breeding analysis offers advantages over other application formats, particularly when analyzing large numbers of samples.

The disclosure provides an amplification, e.g., PCR assay method that comprises obtaining a nucleic acid sample from a B. napus plant, cell, or germplasm thereof, isolating genomic DNA from the sample and screening the isolated DNA for genomic sequence comprising the truncated Ogura Rf segment disclosed herein by contacting the isolated genomic DNA with a restorer forward primer and restorer reverse primer to selectively produce an amplicon comprising part of restorer target sequence, SEQ ID NO:2, and then contacting a labeled probe (restorer probe) to the restorer amplicon, and thereby detecting the restorer amplicon. Such selective amplification will produce an amplicon in proprietary Ogura Rf B. napus lines but will not produce an amplicon in wildtype B. napus lines. The method can further, optionally, include contacting the isolated genomic DNA with a wildtype forward primer and wildtype reverse primer capable of selectively producing a second amplicon of wildtype N19 genomic sequence that includes sequence from the displaced endogenous N19 genomic segment, and then adding a labeled wildtype probe which is capable of detecting the wildtype amplicon. Selective amplification of the wildtype amplicon can be achieved using at least one wildtype primer that anneals within the displaced N19 genomic segment disclosed herein. Such selective amplification will produce an amplicon in wildtype B. napus lines but will not produce an amplicon in proprietary Ogura Rf B. napus lines. The restorer probe and wildtype probe are preferably differently labeled to permit, which can enable the use of both probes in the same reaction mix or in a high throughput amplification assay method. Examples of TaqMan® forward primers, reverse primers, and probes for the detection of the truncated Ogura Rf allele and wildtype genomic allele are provided in Table 2.

TABLE 2

| SEQ ID NO: | Name | Description |
|---|---|---|
| 3 | N101T5R-001-X004 | Restorer VIC Probe |
| 4 | N101T5R-F002 | Restorer Forward Primer 1 |
| 5 | N101T5R-R003 | Restorer Reverse Primer 1 |
| 6 | N101T7A-001-X001 | Wildtype FAM Probe |
| 7 | N101T7A-F001 | Wildtype Forward Primer 2 |
| 8 | N101T7A-R001 | Wildtype Reverse Primer 2 |

The present invention provides a PCR assay method for determining zygosity of a truncated Ogura Rf segment in a B. napus plant, cell or germplasm, the method comprising: (a) performing a first PCR assay using a first probe, a first forward primer, and a first reverse primer on a polynucleotide from a B. napus plant sample, wherein the first probe is SEQ ID NO:3; (b) performing a second PCR assay using a second probe, a second forward primer, and a second reverse primer on the polynucleotide sample, wherein the second probe is SEQ ID NO:6, (c) quantifying the first probe and the second probe; and, (d) comparing the quantified first probe and the quantified second probe of the first PCR assay and the second PCR assay to determine the zygosity.

In an aspect, the probes are detectably labeled. In a further aspect, the first and second probes are labeled with both a fluorescent dye and quencher. In an aspect, the first primers and probes are specific for the truncated Ogura Rf segment in a B. napus plant. In a further aspect, a forward primer specific for the truncated Ogura Rf segment comprises SEQ ID NO: 4, a reverse primer specific for the truncated Ogura Rf segment comprises SEQ ID NO: 5, and a probe specific for the truncated Ogura Rf segment comprises SEQ ID NO: 3. In another aspect, the second primers and probes are specific for the endogenous B. napus N19 sequence in a B. napus plant. In a further aspect, a forward primer specific for the endogenous B. napus N19 sequence comprises SEQ ID NO: 7, a reverse primer specific for the endogenous B. napus N19 sequence comprises SEQ ID NO: 8, and a probe specific for the endogenous B. napus N19 sequence comprises SEQ ID NO: 6. In an aspect, the truncated Ogura Rf segment is absent in a wildtype B. napus plant. In an aspect, the endogenous B. napus N19 sequence is absent in a restorer B. napus plant.

In some aspects, a marker that can determine zygosity of the truncated Ogura Rf segment is marker N101TA9-001-Q001. In some aspects, marker N101TA9-001-Q001 comprises several oligonucleotide primers and probes. In a further aspect, marker N101TA9-001-Q001 comprises the oligonucleotide primers SEQ ID NOs: 4, 5, 7, and 8 and the oligonucleotide probes SEQ ID NOs: 3 and 6. In an aspect, marker N101TA9-001-Q001 detects exogenous radish sequence comprising the truncated Ogura Rf segment and marker N101TA9-001-Q001 detects B. napus sequence in a genomic region displaced by an exogenous radish sequence comprising the truncated Ogura Rf segment. In a further aspect, oligonucleotide primers SEQ ID NOs: 4 and 5 and oligonucleotide probe SEQ ID NO: 3 detect exogenous radish sequence comprising the truncated Ogura Rf segment and do not detect wildtype endogenous B. napus sequence in a genomic region displaced by an exogenous radish sequence comprising the truncated Ogura Rf segment. In another aspect, oligonucleotide primers SEQ ID NOs: 7 and 8 and oligonucleotide probe SEQ ID NO: 6 detect endogenous B. napus sequence in a genomic region displaced by an exogenous radish sequence comprising the truncated Ogura Rf segment and do not detect the exogenous radish sequence comprising the truncated Ogura Rf segment. In a further aspect, the endogenous B. napus sequence is about 2,100 kb long and is at the telomeric end of chromosome N19. In a further aspect, the about 2,100 kb endogenous B. napus sequence has been replaced by the Ogura Rf introgression and is absent in proprietary Ogura Rf germplasm. Therefore, oligonucleotide primers SEQ ID NOs: 7 and 8 and oligonucleotide probe SEQ ID NO: 6 do not detect wildtype B. napus sequence in proprietary Ogura Rf germplasm.

Other methods of detecting the truncated Ogura Rf segment disclosed herein includes single base extension (SBE) methods, which involve the extension of a nucleotide primer that is adjacent to a polymorphism to incorporate a detectable nucleotide residue upon extension of the primer through the polymorphism, e.g., extension through the truncated Ogura Rf segment disclosed herein.

Methods of detecting the truncated Ogura Rf segment disclosed herein also include LCR; and transcription-based amplification methods (e.g., SNP detection, SSR detection, RFLP analysis, and others). Useful techniques include hybridization of a probe nucleic acid to a nucleic acid corresponding to the truncated Ogura Rf segment disclosed herein (e.g., an amplified nucleic acid produced using a genomic B. napus DNA molecule as a template). Hybridization formats including, for example and without limitation, solution phase; solid phase; mixed phase; and in situ hybridization assays may be useful for allele detection in particular embodiments. An extensive guide to hybridization of nucleic acids is discussed in Tijssen, Laboratory Techniques in Biochemistry and Molecular Biology-Hybridization with Nucleic Acid Probes (Elsevier, NY, 1993).

Markers corresponding to genetic polymorphisms between members of a population may be detected by any of numerous methods including, for example and without limitation, nucleic acid amplification-based methods, and nucleotide sequencing of a polymorphic marker region. Many detection methods (including amplification-based and sequencing-based methods) may be readily adapted to high throughput analysis in some examples, for example, by using available high throughput sequencing methods, such as sequencing by hybridization.

The detection of a truncated Ogura Rf segment can be performed by any of a number or techniques, including, but not limited to, the use of nucleotide sequencing products, amplicons, or probes comprising detectable labels. Detectable labels suitable for use include any composition detectable by spectroscopic, radioisotopic, photochemical, biochemical, immunochemical, electrical, optical, or chemical means. Thus, a particular allele of a SNP may be detected using, for example, autoradiography, fluorography, or other similar detection techniques, depending on the particular label to be detected. Useful labels include biotin (for staining with labeled streptavidin conjugate), magnetic beads, fluorescent dyes, radiolabels, enzymes, and colorimetric labels. Other labels include ligands that bind to antibodies or specific binding targets labeled with fluorophores, chemiluminescent agents, and enzymes. In some embodiments of the present invention, detection techniques include the use of fluorescent dyes.

The truncated Ogura Rf segment disclosed herein is associated with a fertility restorer trait. Therefore, any of the methods of detecting the truncated Ogura Rf segment can be used to detect the presence of a fertility restorer trait which is heritable and therefore useful in a breeding program, for example to create progeny B. napus plants comprising the truncated Ogura Rf segment and one or more other desirable agronomic or end use qualities. Accordingly, in some aspects, the invention provides a method of selecting, detecting and/or identifying a B. napus plant, cell, or germplasm thereof (e.g., a seed) having the fertility restorer trait. The method comprises detecting in said B. napus plant, cell, or germplasm thereof, the presence of the truncated Ogura Rf segment and thereby identifying a B. napus plant having the fertility restorer trait.

Breeding Methods with Ogura CMS Fertility Restoration System for B. napus

Hybrid seed production in B. napus that uses the Ogura cytoplasmic male sterility system has two components; a mitochondrial mutation that confers male sterility, and a nuclear restorer fragment (Rf) that restorer's male fertility even in the presence of the mitochondrial mutation. Consequently, a three-line hybrid seed production model comprising a CMS female line (A line), a male fertile maintainer line (B line) which is isogenic to the CMS A line, and a fertility restorer male line (R line) was established. The B line maintains the sterility of the CMS A line and is used to produce the seed of the CMS line by crossing the A×B lines. Hybrids are produced by crossing a R line that contains both Ogura nuclear restorer and sterile cytoplasm, with an A line that is male sterile and contains only the sterile Ogura cytoplasm.

This disclosure provides methods and compositions for selecting a B. napus plant with the truncated Ogura Rf segment and the fertility restorer trait comprising detecting in the plant a molecular marker associated with the truncated Ogura Rf segment. This can be used in a method for selecting such a plant, the method comprises (a) providing a sample of genomic DNA from a B. napus plant; and (b) using any method disclosed herein for detecting in the sample of genomic DNA, the truncated Ogura Rf segment or a genetic marker associated with the truncated Ogura Rf segment.

Methods and compositions of the disclosure may be used to maintain the Ogura cytoplasmic male sterility system. In some aspects, methods and compositions of the disclosure (e.g., the TaqMan® marker N101TA9-001-Q001 assay) are used to confirm: (a) female inbred purity by demonstrating the absence of the truncated Ogura Rf segment in A lines; (b) maintainer line purity by demonstrating the absence of the truncated Ogura Rf segment in B lines; (c) male inbred purity and uniformity or fixity of the truncated Ogura Rf segment by demonstrating homozygous state of the truncated Ogura Rf segment in the male inbred lines; and (d) hybrid purity and uniformity by demonstrating the heterozygous state of the truncated Ogura Rf segment in the hybrids. A low-level presence of a heterozygous Rf state in the male inbred lines can result in segregation into homozygous Rf and homozygous wild types during the seed increase process. Because all males, by design, have sterile Ogura cytoplasm, those plants without Rf become sterile, leading to seed or field discards. This is a frequently encountered problem when males are developed using the pedigree selection method, in new trait development process where males are frequently incorporated with novel variation from other germplasm without Rf, and rarely in trait introgression between B line X R line crosses. All the above issues could result in large scale seed lot discards, delayed product launches, and compromised operational excellence leading to increased cost of goods.

In some aspects, methods and compositions of the disclosure (e.g., the TaqMan® marker N101TA9-001-Q001 assay) may be used for restoring male fertility in B. napus. In an aspect, a method for restoring male fertility in B. napus comprises: (a) crossing a male restorer line that contains both the Ogura nuclear restorer (within the truncated Ogura Rf segment) and sterile cytoplasm and a female line that is male sterile and contains only the sterile Ogura cytoplasm to generate F₁ B. napus plants; (b) using the N101TA9-001-Q001 zygosity assay to determine the F₁ B. napus plant is heterozygous for the truncated Ogura Rf segment and heterozygous for the displaced endogenous N19 genomic sequence; and (c) propagating the identified F₁ B. napus plant, thereby restoring male fertility in B. napus. In a further aspect, the truncated Ogura Rf segment is amplified with oligonucleotide primers SEQ ID NOs: 4 and 5 and detected with oligonucleotide probe SEQ ID NO: 3. In a further aspect, the displaced endogenous N19 genomic sequence is amplified with oligonucleotide primers SEQ ID NOs: 7 and 8 and detected with oligonucleotide probe SEQ ID NO: 6.

This disclosure also provides a method comprising the transfer by introgression of the truncated Ogura Rf segment from one plant into a recipient plant by various cross pollination and selection methods. This transfer can be accomplished using, for example, standard crossing, backcrossing, forward breeding, and selection techniques to reduce the stature of the recipient plant and/or the progeny of the recipient plant. In one aspect, the truncated Ogura Rf segment is introgressed into one or more restorer B. napus lines which will be used for the development of commercial or elite B. napus varieties using marker-assisted selection (MAS) or marker-assisted breeding (MAB). MAS and MAB involve the use of one or more molecular markers that indicate the presence or co-segregation with the truncated Ogura Rf segment and used for the identification and selection of those offspring plants that contain the truncated Ogura Rf segment.

The truncated Ogura Rf segment, once established, can be transferred through introgression into other plants within the same Brassica napus, Brassica campestris, or Brassica juncea species by conventional plant breeding techniques involving cross-pollination and selection of the progeny (i.e., MAS or MAB). The restorer gene is highly heritable, can be transmitted to progeny, and can be recovered in segregating progeny in subsequent generations following crossing. Also, once established the desired trait can be transferred between the B. napus, B. campestris, and B. juncea species using the same conventional plant breeding techniques involving pollen transfer and selection. The transfer of traits between Brassica species, such as B. napus and B. campestris, by standard plant breeding techniques is already well documented in the technical literature. (See, for instance, Tsunada et al., 1980).

When a population is segregating for multiple loci affecting one or multiple traits, e.g., multiple loci involved in resistance to a single disease, or multiple loci each involved in resistance to different diseases, the efficiency of MAS compared to phenotypic screening becomes even greater because all the loci can be processed in the lab together from a single sample of DNA. Thus, MAS is particularly suitable for introgressing the truncated Ogura Rf segment into a plant line that includes one or more additional desirable traits. Additional desirable traits can include, but are not limited to, herbicide resistance, insect resistance; resistance to bacterial, fungal, or viral disease; male fertility, male sterility, enhanced nutritional quality, enhanced oil quality, industrial usage, yield stability, or yield enhancement.

Another use of MAS in plant breeding is to assist the recovery of the recurrent parent genotype by backcross breeding. Backcross breeding is the process of crossing a progeny back to one of its parents. Backcrossing is usually done for the purpose of introgressing one or a few loci from a donor parent, i.e., truncated Ogura Rf segment, into an otherwise desirable genetic background from the recurrent parent. The more cycles of backcrossing that are done, the greater the genetic contribution of the recurrent parent to the resulting variety. This is desirable when the recurrent parent is an elite variety and/or has more desirable qualities than the donor plant, even though the recurrent parent may need a fertility restorer trait. For example, backcrossing can be desirable when a recurrent plant provides better yield, fecundity, disease and/or insect resistance, and the like, as compared to the donor truncated Ogura Rf segment plant.

Thus, traditional breeding techniques can be used to introgress a nucleic acid sequence associated with truncated Ogura Rf segment into a recipient B. napus plant. For example, inbred restorer B. napus plant lines comprising the truncated Ogura Rf segment can be developed using the techniques of recurrent selection and backcrossing, selfing, or any other technique used to make parental lines. In a method of recurrent selection and backcrossing, the truncated Ogura Rf segment can be introgressed into a target recipient plant (the recurrent parent) by crossing the recurrent parent with a first donor plant, which differs from the recurrent parent and is referred to herein as the “non-recurrent parent.” The recurrent parent is a plant, in some cases, comprises commercially desirable characteristics, such as, but not limited to disease and/or insect resistance, valuable nutritional characteristics, valuable abiotic stress tolerance, and the like. The non-recurrent parent can be any plant variety or inbred line that is cross-fertile with the recurrent parent.

The resulting progeny plant population is then screened for the desired characteristics, including the truncated Ogura Rf segment, which screening can occur in a number of different ways. For instance, the progeny population can be screened using phenotypic pathology screens or quantitative bioassays as are known in the art. Alternatively, instead of using bioassays, MAS or MAB can be performed using one or more of methods and compositions described herein to identify progeny plants or germplasm that comprise a truncated Ogura Rf segment. Also, MAS or MAB can be used to confirm the results obtained from the quantitative bioassays. The markers, primers, and probes described herein can be used to select progeny plants by genotypic screening.

Following screening, the F₁ progeny (e.g., hybrid) plants having the truncated Ogura Rf segment can be selected and backcrossed to the recurrent parent for one or more generations in order to allow for the B. napus plant to become increasingly inbred. This process can be repeated for one, two, three, four, five, six, seven, eight, or more generations. In some examples, the recurrent parent plant or germplasm used in this method is an elite B. napus variety. Thus, this crossing and introgression method can be used to produce a progeny B. napus plant or germplasm having the truncated Ogura Rf segment introgressed into a genome that is at least about 50%, 55%, 60%, 65%, 70%, 75%, 80%, 85%, 90%, 95%, 97%, 99% identical to that of the elite B. napus variety.

Also provided is a method of producing a plant, cell, or germplasm (e.g., seed thereof) that comprises crossing a first B. napus plant or germplasm with a second B. napus plant or germplasm, wherein said first B. napus plant or germplasm comprises within its genome a truncated Ogura Rf segment disclosed herein, collecting seed from the cross and growing a progeny B. napus plant from the seed, wherein said progeny B. napus plant comprises in its genome said truncated Ogura Rf segment, thereby producing a progeny plant that carries the segment associated with the fertility restorer trait disclosed herein.

In some aspects, the TaqMan® marker N101TA9-001-Q001 is used in the method to introgress the truncated Ogura Rf segment into a B. napus plant. In an aspect of the present invention, a method of introducing a truncated Ogura Rf segment into a B. napus plant comprises: (a) crossing a first parent B. napus plant comprising a truncated Ogura Rf segment on chromosome N19 with a second parent B. napus plant that does not have the truncated Ogura Rf segment to produce hybrid progeny plants; and (b) obtaining a nucleic acid sample from one or more hybrid progeny plants; and (c) selecting the one or more hybrid progeny plants having the truncated Ogura Rf segment using the N101TA9-001-Q001 zygosity assay described herein. The method further comprises: (d) crossing the one or more selected progeny plants with the first or second parent B. napus plant (the recurrent parent plant) to produce backcross progeny plants; (e) obtaining a nucleic acid sample from one or more backcross progeny plants; and (f) selecting the one or more backcross progeny plants having the truncated Ogura Rf segment using the N101TA9-001-Q001 zygosity assay described herein to produce another generation of backcross progeny plants. The method further comprises repeating steps (d), (e), and (f) three or more times to produce backcross progeny plants that comprise the truncated Ogura Rf segment and the agronomic characteristics of the recurrent parent plant when grown in the same environmental conditions. In a further aspect, the Ogura Rf segment is amplified with oligonucleotide primers SEQ ID NOs: 4 and 5 and detected with oligonucleotide probe SEQ ID NO: 3. In a further aspect, the displaced endogenous B. napus genomic sequence is amplified with oligonucleotide primers SEQ ID NOs: 7 and 8 and detected with oligonucleotide probe SEQ ID NO: 6.

While the invention has been particularly shown and described with reference to a preferred embodiment and various alternate embodiments, it will be understood by persons skilled in the relevant art that various changes in form and details can be made therein without departing from the spirit and scope of the invention. For instance, while the particular examples below may illustrate the methods and embodiments described herein using a specific plant, the principles in these examples may be applied to any plant. Therefore, it will be appreciated that the scope of this invention is encompassed by the embodiments of the inventions recited herein and, in the specification, rather than the specific examples that are exemplified below. All cited patents and publications referred to in this application are herein incorporated by reference in their entirety, for all purposes, to the same extent as if each were individually and specifically incorporated by reference.

### EXAMPLES

The following are examples of specific embodiments of some aspects of the invention. The examples are offered for illustrative purposes only and are not intended to limit the scope of the invention in any way. Efforts have been made to ensure accuracy with respect to numbers used (e.g., amounts, temperatures, etc.), but some experimental error and deviation should, of course, be allowed for.

### Example 1: Sequencing Characterization of a Truncated Ogura Rf Segment

A truncated version of the Ogura Rf introgression has been developed by shortening the radish introgression via gamma ray mutagenesis and therefore eliminating some linkage drag. The truncated Ogura Rf segment has been characterized in three proprietary male restorer (R) line reference genomes: CANOLA4_G00555MC.CHROMOSOMES (G00555MC), CANOLA2_N00655MC.CHROMOSOMES (N00655MC), and NW2236MC_V2 (NW2236MC). A list of relevant sequenced genomes is described in Table 3. The G00555MC and N00655MC reference genomes were created internally using Bionano optical mapping (Bionano Genomics, San Diego, CA) and PacBio long read sequencing (PacBio, Menlo Park, CA). NW2236MC reference genome was created via collaboration with NRGene (San Diego, CA) using their PanMAGIC platform.

TABLE 3

| Full Name | Abbreviated Name | Source | Description |
|---|---|---|---|
| CANOLA4_G00555MC. | G00555MC | PHI | restorer line |
| CHROMOSOMES | | | |
| CANOLA2_N00655MC. | N00655MC | PHI | restorer line |
| CHROMOSOMES | | | |
| NW2236MC_V2 | NW2236MC | PHI/NRGene | restorer line |
| Canola3_G00010BC. | G00010BC | PHI | B-line |
| CHROMOSOMES | | | |
| NS1822BC.GOLD. | NS1822BC | PHI | B-line |
| CHROMOSOMES_v2 | | | |
| Radish_v1.0 | Radish_v1.0 | Public | public radish line |
| NRGene N99 genome | N99 | NRGene | wt B. napus (no radish |
| | | | introgression) |

Based on previous mapping results, the Ogura Rf introgression is known to be located at the telomeric end of N19. The first 10 Mb of the N19 chromosome in all three male lines were compared to the NS1822BC.GOLD.CHROMOSOMES_v2 (NS1822BC) genome as well as the Radish_v1.0 genome using whole genome alignment algorithm MAUVE (Darling A C et al. 2004. Genome Res. 14 (7): 1394-403). The breakpoint between the B. napus DNA and radish DNA was identical in the two internal reference genomes, G00555MC and N00655MC. The length of the radish introgression in both G00555MC and N00655MC was found to be 4,940,398 bp. This 4.9 Mb correlates to the 5.4 Mb sequence on chromosome R9, Radish v1.0_R9: 21,884,425-27,337,721. The radish introgression in the restorer lines is not 100% colinear with the public radish assembly.

The NRGene reference assembly of NW2236MC does not have the radish DNA found on chromosome N19. Instead, the N19 chromosome begins just after the radish segment. The assembly was created by anchoring to the NRGene N99 genome, which does not have the radish introgression. Therefore, the segment was not anchored to the N19 chromosome, and instead exists as part of the unmapped scaffolds of the genome.

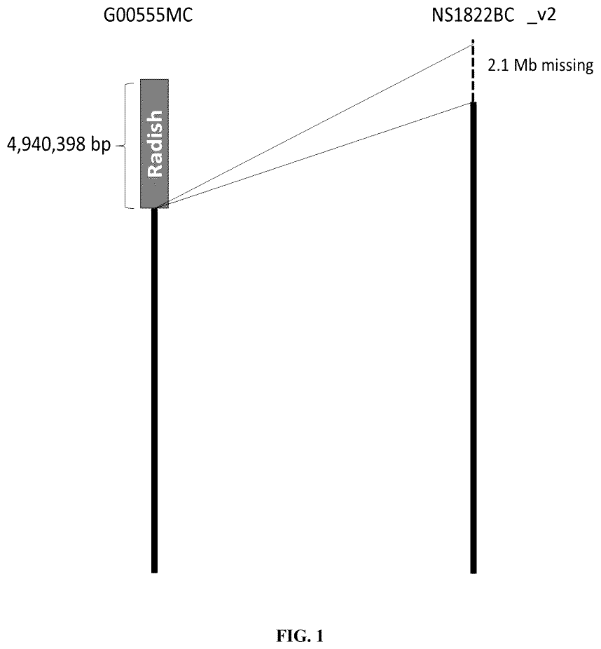

The junction between the radish introgression and B. napus genome was identified in the reference genome G00555MC, which was then compared to a B-line reference genome, NS1822BC, and the radish public reference assembly, Radish_v1.0. The G00555MC breakpoint consists of radish DNA, then a small segment (˜400 bp) of B. napus DNA that has been translocated and inverted from upstream on N19. The B. napus N19 chromosome then continues, starting at about 2,100 kb in the NS1822BC genome. The truncated Ogura Rf segment therefore includes a deletion of about 2,100 kb of B. napus DNA (FIG. 1).

### Example 2: Restorer Zygosity Assay Development

Reference genomes for two R-lines, G00555MC and N00655MC, which contain truncated Ogura Rf segment, and for two B-lines, G00010BC and NS1822BC, were developed. By comparatively analyzing orthologous N19 chromosomal region containing Ogura Rf segment between these R and B lines, along with publicly available diploid radish orthologous chromosome R9 sequence, it was possible to precisely characterize the attributes of the truncated Ogura Rf segment, including identification of introgression break points at base pair level. A TaqMan® assay was developed that detects the Ogura Rf segment and its zygosity state at a low cost and fast turnaround time on single seed or leaf tissue from a single plant.

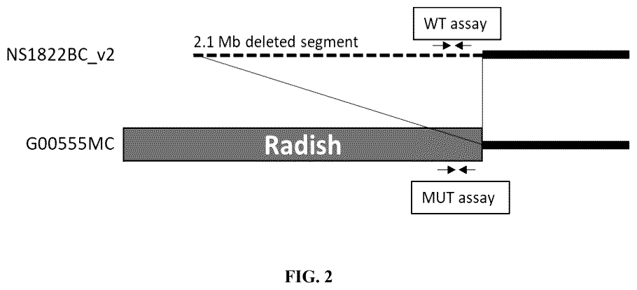

The wildtype (non-Rf) target sequence located on NS1822BC at N19: 1910704-1910716 bp (SEQ ID NO:1) and the mutant (Rf) target sequence (SEQ ID NO:2) located on G00555MC at N19: 4939371-4939478 bp were used as the target sequence for the PCR assay design. The wildtype N19 sequence is present in B. napus A and B lines but absent in R lines. The truncated Ogura Rf segment is present in the R lines but absent in the A and B lines. Marker N101TA9-001-Q001 was developed upstream from the breakpoint where the Ogura Rf segment introgressed on N19 (FIG. 2). The wildtype specific primers and probe were designed in the about 2,100 kb deletion of B. napus DNA (FIG. 2). Marker N101TA9-001-Q001 primers and probes are described in Tables 1 and 2.

N101TA9-001-Q001 was validated on a diverse set of inbred and hybrid B. napus lines with North American, Australian, and European origin. The set included over 750 B. napus inbred A, B, and R lines and over 50 hybrid lines. N101TA9-00-Q001 had 97% concordance with the expected restorer phenotype on the panel of diverse set of inbred and hybrid B. napus lines. The marker was validated further in the plate format and the Array Tape format described in Example 3. There were 252 common individuals between the two panels.

### Example 3: DNA Extraction and Marker Amplification Protocols

Plate format for marker development. DNA was extracted using the CTAB method. Briefly, genomic DNA was extracted from 8, 2 mm fresh leaf disks that were lyophilized 24 hours. The leaf disks were collected into 0.5 ml tubes and placed in 96 well deep well plates. These plates were pressed with a pneumatic press and two BBs were added to each tube. A paper blotter pad was secured to the top of the plates with tape and the plates were pressed again to form a seal. The tissue was then disrupted with a GenoGrinder® (SPEX® SamplePrep, Metuchen, NJ) for two minutes at 1450 rpm. The blotter pads were removed and 450 μl of CTAB extraction buffer (1M Tris HCl, 0.5M EDTA, 1M CTAB powder, 5M NaCl, and water) was added to each tube using a Biomek uFill (Beckman Coulter, Pasadena, CA). The plates were sealed with a heat sealer, shook by hand for 30 seconds and incubated for one hour at 65° C. The seals were removed from the plate and 300 μl of a 24:1 chloroform: octanol solution was added. The plates were re-sealed, inverted by hand for 30 seconds and spun in a centrifuge at 3500 rpm for 25 minutes at 4° C. The seals were removed, and the supernatant was transferred to a new deep well plate containing 175 μl of isopropanol. The new plates were sealed, inverted by hand 10 times and incubated at −20° C. for 90 minutes, then centrifuged at 3500 rpm for 25 minutes at 4° C. The seal was removed, and the isopropanol was decanted away. The plates were dried overnight at room temperature. The DNA was re-suspended in 50 μl 8V TE. The DNA was diluted 1:100 for PCR amplification.

Primers and probes were combined for a final assay concentration of 18 μM of each probe, and 4 μM of each primer. 13.6 μl of the assay was combined with 1000 μl of LGC BHQ Probe Master Mix (LGC, Biosearch Technologies, Middleton, WI). A Meridian liquid handler (LGC, Biosearch Technologies) dispensed 1.3 μl of the mix onto a 1536 plate containing ˜6 ng of dried DNA. The plate was sealed with a Phusion laser sealer (LGC, Biosearch Technologies) and thermocycled using a Hydrocycler (LGC, Biosearch Technologies) with the following conditions: 94° C. for 10 min, 40 cycles of 94° C. for 30 sec, 60° C. for 1 min. The excitation at wavelengths 485 (FAM) and 520 (VIC) was measured with a Pherastar plate reader (BMG LabTech, Cary, NC). The values were normalized against ROX and plotted and scored on scatterplots utilizing the KRAKEN software (LGC, Biosearch Technologies).

Array tape format for production validation of marker assays. Genomic DNA was extracted from 2, 2 mm fresh leaf disks that were lyophilized 24 hours. The leaf disks were collected into 0.5 ml tubes and placed in 96 well deep well plates. These plates were pressed with a pneumatic press and two BBs were added to each tube. 350 μl of HotShot extraction buffer (25 mM NaOH and 0.2 mM sodium calcium EDTA) was added to each tube using a Biomek uFill. The plates were sealed with a heat sealer, and the tissue disrupted with a GenoGrinder® for one minute at 1450 rpm. The plates were then incubated for 30 minutes at 95° C. The plates were cooled to the touch and then centrifuged at 3300 g for two minutes. DNA was diluted 1:4 with dilution buffer (2:1 ratio of neutralization buffer and TE) for use in PCR genotyping.

Primers and probes were synthesized by Life Technologies (Carlsbad, CA). 40× assays were diluted to 20× (1:1 dilution) for use in PCR genotyping and dispensed into single use tubes. 278 μL LGC BHQ Probe Master Mix was added to a single use tube and centrifuged at 250 g for one minute. Marker and master mix were then dispensed using a Nexar® In-Line Liquid Handling and Assay Processing System, onto a 384-well-Array Tape (Douglas Scientific, Alexandria, MN). The Array Tape was then rewound and sealed. Sealed Array Tapes were thermocycled using a Soellex™ (LGC-Biosearch Technologies, Alexandria, MN) with the following conditions: 94° C. for 10 min, 40 cycles of 94° C. for 30 sec, 60° C. for 1 min. After the thermocycling was completed, the Array Tape was dried using a centrifuge at 240 rpm for 35 minutes at an air pressure of 90 psi. The excitation at wavelengths 485 (FAM) and 520 (VIC) was measured with an Arraya Inline Fluorescence Detection System (LGC-Biosearch Technologies). The values were normalized against ROX and plotted and scored on scatterplots utilizing proprietary software.

### Example 4: Restorer Zygosity Assay Validation

Restorer B. napus lines comprising the truncated Ogura Rf segment described herein are developed from restorer B. napus lines comprising a shortened Raphanus fragment (SRF) described in U.S. Pat. No. 8,466,347B2. These SRF lines are deposited at the NCIMB (National Collections of Industrial, Marine and Food Bacteria NCIMB Ltd, Ferguson Building, Craibstone Estate, Bucksburn, Aberdeen, AB21 9YA. Scotland, UK).

The restorer zygosity assay, N101TA9-001-Q001, is used to validate the presence or absence of the about 4,900 kb truncated Ogura Rf segment and the presence or absence of the about 2,100 kb wildtype B. napus N19 genomic sequence in the SRF restorer lines. The restorer assay is performed using the methods described in Example 3 and the primers and probes listed in Tables 1 and 2.

The results of the assay show the presence of two copies of the truncated Ogura Rf segment and zero copies of the wildtype N19 sequence in at least one of the SRF lines described in U.S. Pat. No. 8,466,347B2.

We claim:

1. A method of identifying a B. napus plant, cell, or germplasm thereof comprising a truncated Ogura Rf segment that includes the Rfo gene, the method comprising:
a. obtaining a nucleic acid sample from a B. napus plant, cell, or germplasm; and
b. screening the sample for the truncated Ogura Rf segment on chromosome N19, wherein the segment is less than about 5,000 kb in length, and wherein the presence of the segment contributes to a fertility restorer phenotype in B. napus.

2. The method of claim 1, wherein the method further comprises:
c. selecting a B. napus plant, cell, or germplasm that comprises the truncated Ogura Rf segment that is less than about 5,000 kb in length.

3. The method of claim 1, wherein the method further comprises screening for the presence of a displaced endogenous B. napus genomic segment on the telomeric end of chromosome N19, wherein the genomic segment is about 2,100 kb, and wherein the presence of the genomic segment contributes to a wildtype phenotype in B. napus.

4. The method of claim 1, wherein screening for the presence of the truncated Ogura Rf segment or the presence of the displaced N19 genomic segment comprises DNA sequencing.

5. The method of claim 1, wherein the method further comprises:
a. contacting the isolated nucleic acid sample with a restorer forward primer and restorer reverse primer to selectively produce an amplicon that includes sequence from the truncated Ogura Rf segment, and
b. optionally, contacting the isolated nucleic acid sample with a wildtype forward primer and wildtype reverse primer to selectively produce a second amplicon that includes sequence from the displaced wildtype N19 genomic segment.

6. The method of claim 5, wherein the restorer forward primer comprises SEQ ID NO:4, the restorer reverse primer comprises SEQ ID NO:5, the wildtype forward primer comprises SEQ ID NO:7, and the wildtype reverse primer comprises SEQ ID NO:8.

7. The method of claim 6, wherein the method further includes
a. contacting the amplicon with a restorer probe to detect amplified genomic sequence from the truncated Ogura Rf fragment; and
b. optionally, contacting the second amplicon with a wildtype probe to detect amplified genomic sequence from the displaced wildtype N19 genomic segment.

8. The method of claim 6, wherein the restorer probe comprises SEQ ID NO:3 and the wildtype probe comprises SEQ ID NO:6.

9. A method of introgressing a fertility restorer trait into a B. napus plant comprising:
a. crossing a first parent B. napus plant comprising a truncated Ogura Rf segment with a second parent B. napus plant that does not have the segment to produce hybrid progeny plants; and
b. obtaining a nucleic acid sample from one or more hybrid progeny plants; and
c. selecting the one or more hybrid progeny plants having the truncated Ogura Rf segment in accordance with the method of claim 1.

10. The method of claim 9 further comprising:
d. crossing the one or more selected progeny plants with the first or second parent B. napus plant (the recurrent parent plant) to produce backcross progeny plants;
e. obtaining a nucleic acid sample from one or more backcross progeny plants; and
f. selecting the one or more backcross progeny plants having the truncated Ogura Rf segment to produce another generation of backcross progeny plants.

11. The method of claim 10 further comprising:
g. repeating steps (d), (e), and (f) three or more times to produce backcross progeny plants that comprise the truncated Ogura Rf segment and the agronomic characteristics of the recurrent parent plant when grown in the same environmental conditions.

12. The introgressed B. napus plant produced by the method according to claim 9.

13. A method for restoring male fertility in B. napus, the method comprising:
a. crossing a male restorer line that contains both the truncated Ogura Rf segment and a female line that is male sterile and does not contain the truncated Ogura Rf segment to generate F₁ B. napus plants;
b. selecting the F₁ B. napus plants having the truncated Ogura Rf segment in accordance with the method of claim 1; and
c. propagating the identified F₁ B. napus plant, thereby restoring male fertility in the B. napus plant.

14. The PCR assay method of claim 1, wherein the method is used to confirm:
a. female inbred purity by demonstrating the absence of the truncated Ogura Rf segment in A lines;
b. maintainer line purity by demonstrating the absence of the truncated Ogura Rf segment in B lines;
c. male inbred purity and uniformity or fixity of the truncated Ogura Rf segment by demonstrating homozygous state of the truncated Ogura Rf segment in the male inbred lines; and
d. hybrid purity and uniformity by demonstrating the heterozygous state of the truncated Ogura Rf segment in the hybrids.

15. A PCR assay method for determining zygosity of a truncated Ogura Rf segment in a B. napus plant, cell or germplasm, the method comprising:
a. performing a first PCR assay using a first probe, a first forward primer, and a first reverse primer on a polynucleotide from a B. napus plant sample, wherein the first probe is SEQ ID NO:3;
b. performing a second PCR assay using a second probe, a second forward primer, and a second reverse primer on the polynucleotide sample, wherein the second probe is SEQ ID NO:6;
c. quantifying the first probe and the second probe; and,
d. comparing the quantified first probe and the quantified second probe of the first PCR assay and the second PCR assay to determine the zygosity.

16. The method of claim 15, wherein the first probe detects the presence of the truncated Ogura Rf segment, and the second probe detects the displaced wildtype N19 genomic segment.

17. The method of claim 15, wherein the first forward primer comprises SEQ ID NO:4, the first reverse primer comprises SEQ ID NO:5, second forward primer comprises SEQ ID NO: 7, and second reverse primer comprises SEQ ID NO:8.

18. The method of claim 15, wherein the PCR assay method comprises a TaqMan, KASP, gel-based assay, or sequencing based assay.

19. The method of claim 18, wherein the PCR assay method comprises a TaqMan zygosity assay.

20. A homozygous restorer B. napus plant, wherein the plant comprises the truncated Ogura Rf segment that is about 4,900 kb and does not comprise the wildtype N19 genomic segment that is about 2,100 kb.


SEQUENCE LISTING (8 sequences)

SEQ ID NO: 1 - DNA, 413 bp, Brassica napus
  source 1..413: mol_type = unassigned DNA
cattttcgtccggcggaattcgagcaaaagggtctgtcttctctccgtaaatctagtgtctttttcaacttatccgttataattctcctccgaaatctctgggtccttccgatcaatttcgagctttgggtttctcaatttcgatgatgggtattgcgaaagtttcgaaatttgacgaaaaaattgagtattcattggctgattagggtttgcgattcaatttgttctggtttagcttcgatttggttgttttggactctaaaatgatcgaaaacaacaaaaagatagaaactttagtgttaaagattagatgtgatgaatgtgttttaatctctatgtttgtattgattcagctagattttttggaactatggagagcttctggcaattgggtgacgagctccgaggtcagt

SEQ ID NO: 2 - DNA, 249 bp, Raphanus sativus
  source 1..249: mol_type = unassigned DNA
tgaggtaatacttcctccacaaatcgttactttcaattcgtagtttttttttttgtctttgtgtttccggttgatttgcatgtttgtttgatgaaattgagttttccttgtccgagtctgagatctcgatcgatgttatgtatgtcttggaaattgagttctttcagtttatttgttgtgtgtttctttttaagtatatatctgaaaccgatggagttagttcgtgtcggtggtgaaggacttaggttt

SEQ ID NO: 3 - DNA, 21 bp, synthetic construct
  source 1..21: mol_type = other DNA
atctcagactcggacaaggaa

SEQ ID NO: 4 - DNA, 24 bp, synthetic construct
  source 1..24: mol_type = other DNA
tgaaagaactcaatttccaagaca

SEQ ID NO: 5 - DNA, 20 bp, synthetic construct
  source 1..20: mol_type = other DNA
ttgtgtttccggttgatttg

SEQ ID NO: 6 - DNA, 16 bp, synthetic construct
  source 1..16: mol_type = other DNA
tcattggctgattagg

SEQ ID NO: 7 - DNA, 20 bp, synthetic construct
  source 1..20: mol_type = other DNA
cgatgatgggtattgcgaaa

SEQ ID NO: 8 - DNA, 24 bp, synthetic construct
  source 1..24: mol_type = other DNA
cgaagctaaaccagaacaaattga